(19) United States
(12) Patent Application Publication
(10) Pub. No.: US 2025/0041545 A1
(43) Pub. Date: Feb. 6, 2025

(54) INLINE MUFFLER AND POSITIVE AIRWAY PRESSURE THERAPY APPARATUS INCLUDING SAME

(71) Applicant: SOMNETICS INTERNATIONAL, INC., Fridley, MN (US)

(72) Inventor: Brian W. Ward, Littleton, CO (US)

(21) Appl. No.: 18/760,681

(22) Filed: Jul. 1, 2024

Related U.S. Application Data
(60) Provisional application No. 63/529,845, filed on Jul. 31, 2023.

(51) Int. Cl. A61M 16/00; A61M 16/06; A61M 16/08

(52) U.S. Cl. CPC A61M 16/0003; A61M 16/0066; A61M 16/0605; A61M 16/0875; A61M 2205/42; A61M 2210/0618; A61M 2210/0625

(57) ABSTRACT

A muffler for use in a positive airway pressure system includes a housing with an inlet port adapted to operatively couple to receive a pressurized gas flow, an outlet port, and a tubular body extending between the inlet port and the outlet port. The muffler also includes a muffler insert positioned within the tubular body in an abutting relationship with an inner surface of the body. The muffler insert defines: a first chamber proximate the inlet port; a second chamber proximate the outlet port; and an intermediate chamber separate from, and located between, the first and second chambers. First and second pluralities of apertures are adapted to direct the pressurized gas flow between adjacent chambers. The muffler attenuates noise associated with the pressurized gas flow as the pressurized gas flow passes through the muffler.

### RELATED APPLICATION

This application claims priority to U.S. Provisional Patent Application Ser. No. 63/529,845, filed Jul. 31, 2023. The entire disclosure of this application is hereby incorporated herein by reference in its entirety.

Embodiments of the present disclosure relate to positive airway pressure systems and, more particularly, to inline mufflers for use with the same.

### BACKGROUND

Positive airway pressure (PAP) therapies are frequently used in the treatment of, among other ailments, obstructive sleep apnea, complex sleep apnea, asthma, bronchitis, chronic obstructive pulmonary disease (COPD), snoring, and congestive heart failure. These therapies typically provide a flow of pressurized gas (e.g., typically air, but may be most any gas or gas-vapor mixture including, for example, oxygen and medicinal vapors) to pressurize the airway of a user to a pressure in the range of 4-30 centimeters (cm) of water (H₂O) (e.g., often about 4-20 cm H₂O) or more. Depending upon the particular therapy, a variable or a constant pressure therapy may be administered to the user to reduce or eliminate airway occlusions (or to otherwise treat acute or chronic respiratory failure) that necessitated the use of the therapy.

Regardless of the particular therapy, positive airway pressure apparatus typically includes at least a blower unit and a user interface. A delivery tube or hose may also be included to connect the blower unit to the user interface, wherein the hose and interface may together define a delivery conduit. The blower unit may rest on a bedside table or floor adjacent the bed (or in the bed), or alternatively, may attach to the user. The blower may typically include a fan or impeller connected to an output shaft of a motor. A controller regulates the motor to control fan speed and thus therapy pressure. The user interface is configured to be secured relative to the user's head in such a way as to form a generally air-tight seal with the user's airway. As a result, the fan may generate a flow of pressurized gas that is delivered to the airway via the delivery conduit.

### SUMMARY

Embodiments described herein include a muffler for use in a positive airway pressure system. The muffler includes a housing defined by two halves. The housing defines: an inlet port adapted to operatively couple to receive a pressurized gas flow; an outlet port adapted to output the pressurized gas flow; and a tubular body extending between the inlet port and the outlet port. The muffler also includes a muffler insert positioned within the tubular body in an abutting relationship with an inner surface of the body. The muffler insert defines: a first chamber proximate the inlet port; a second chamber proximate the outlet port; and an intermediate chamber separate from, and located between, the first and second chambers. The insert also includes a first wall separating the first chamber from the intermediate chamber; and a second wall separating the second chamber from the intermediate chamber. The first and second walls respectively define first and second pluralities of apertures adapted to direct the pressurized gas flow between adjacent chambers. The muffler is adapted to attenuate noise associated with the pressurized gas flow as the pressurized gas flow passes through the muffler.

In another embodiment, a method involves receiving a pressurized gas flow at an inlet port of a muffler insert for use in a positive airway pressure system. The pressurized gas flow is sent from the inlet port to, in order: a first chamber proximate the inlet port; an intermediate chamber; and a second chamber proximate an outlet port of the muffler insert. The method further involves attenuating noise associated with the pressurized gas flow via: first apertures between the first chamber and the intermediate chamber; and second apertures between the intermediate chamber and the second chamber.

The above summary is not intended to describe each embodiment or every implementation. Rather, a more complete understanding of illustrative embodiments will become apparent and appreciated by reference to the following Detailed Description of Exemplary Embodiments and claims in view of the accompanying figures of the drawing.

### BRIEF DESCRIPTION OF THE VIEWS OF THE DRAWING

In the following detailed description of illustrative embodiments, reference is made to the accompanying figures of the drawing which form a part hereof, and in which are shown, by way of illustration, specific embodiments that may be practiced.

FIG. 1 is a perspective view of a positive airway pressure apparatus including an inline muffler in accordance with embodiments of the present disclosure, the muffler located between a blower and a user interface (e.g., mask);

FIG. 2 is a side elevation view of the apparatus of FIG. 1;

FIG. 3 is an exploded perspective view of portions of the apparatus of FIG. 1;

FIG. 4 is an isolated perspective view of the muffler of FIGS. 1-3;

FIG. 5 is an exploded perspective view of the muffler of FIGS. 1-4;

FIG. 6 is a perspective view of halves of a muffler insert according to an example embodiment;

FIG. 7. is a perspective view of the muffler insert shown in FIG. 6 in an assembled configuration;

FIGS. 8 and 9 are cross sectional views of the muffler insert as shown in FIG. 7;

FIGS. 10 and 11 are cross sectional views of a muffler insert according to one or more alternate embodiments;

FIG. 12 is a side view of a muffler housing according to one or more alternate embodiments;

FIG. 13 is a block diagram of a positive airway pressure apparatus according to an example embodiment; and

FIG. 14 is a flowchart of a method according to an example embodiment.

The figures are rendered primarily for clarity and, as a result, are not necessarily drawn to scale. Moreover, various structure/components, including but not limited to fasteners, electrical components (wiring, cables, etc.), and the like, may be shown diagrammatically or removed from some or all of the views to better illustrate aspects of the depicted embodiments, or where inclusion of such structure/components is not necessary to an understanding of the various exemplary embodiments described herein. The lack of illustration/description of such structure/components in a particular figure is, however, not to be interpreted as limiting the scope of the various embodiments in any way.

### DETAILED DESCRIPTION OF EXEMPLARY EMBODIMENTS

In the following detailed description of illustrative embodiments, reference is made to the accompanying figures of the drawing which form a part hereof, and in which are shown, by way of illustration, specific embodiments that may be practiced.

Embodiments described herein are directed generally to positive airway pressure apparatus, systems, and methods and, more particularly, to inline mufflers for use with the same. While described herein primarily in the context of treatment of sleep-disordered breathing, those of skill in the art will realize that the same or similar embodiments are applicable to most any assisted respiration or ventilation system, and in fact to most any positive airway pressure apparatus/system. Variations, combinations, and modifications of the embodiments described herein will be apparent to those skilled in the art, and it should be understood that this disclosure is not limited to the illustrative embodiments set forth herein.

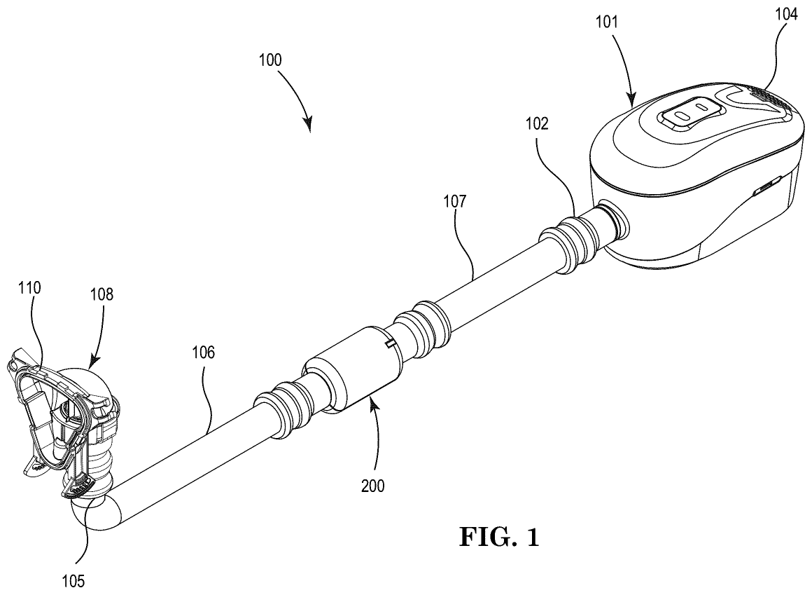

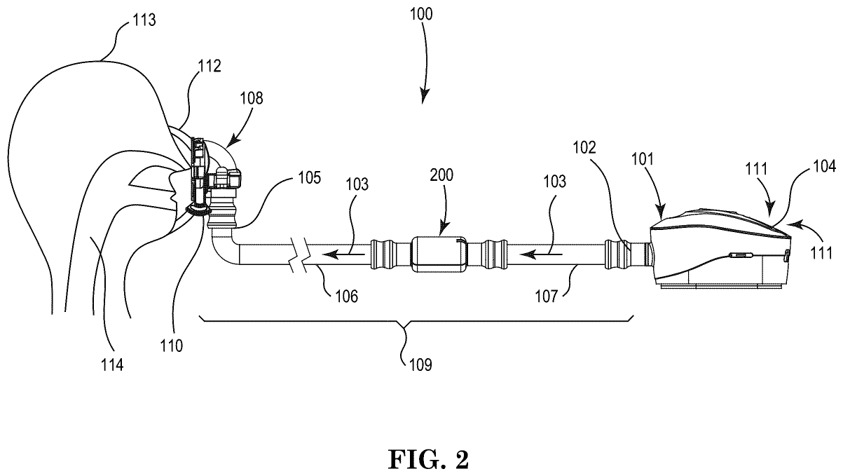

With reference to the drawing, wherein like reference numerals designate like parts and assemblies throughout the several views, FIGS. 1 and 2 illustrate an exemplary, non-invasive, positive airway pressure (PAP) apparatus 100. The PAP apparatus 100 may include a flow generator forming a housing containing a blower 101 adapted to generate or otherwise produce a flow of pressurized gas 103 at a blower outlet 102. The outlet 102 is operatively coupled to and in fluid communication with a first or proximal end of an elongate delivery hose or tube 106 (via an intermediate hose 107 and muffler 200 as described below). A second or distal end of the tube 106 may be connected to an inlet 105 of a user interface 108.

The user interface 108 may include a frame 110 adapted to support a flexible seal 112 (not shown in FIG. 1 but shown diagrammatically in FIG. 2). The user interface 108 may include most any structure that seals effectively to a user 113 (e.g., to the user's face) in such a way that pressurized gas delivered to the user interface may be communicated to an airway 114 of the user without excessive unintentional gas leakage. For example, the user interface could be a face mask that covers one or both of the user's mouth and nose; a nares pillow seal; an intubation tube; or any similar device. For simplicity, the user interface may be referred to herein simply as a “mask” 108 without limitation.

As used herein, the terms “air,” “gas,” and “fluid” are understood to include most any gas or gas-vapor combination. For example, the gas provided by the blower may include ambient air, oxygen, water vapor, medicinal vapor, and combinations thereof. For simplicity, the terms air, fluid, and gas may, unless otherwise indicated, be used interchangeably herein without limitation.

The tube 106 and user interface 108 may together define a portion of a gas delivery path or delivery conduit 109 (see FIG. 2) adapted to provide or communicate a flow of pressurized gas from the blower 101 to the airway 114 of the user 113. The delivery conduit 109 may include one or more vents or ports to provide what is referred to as an “intentional leak” or “intentional vent leak.” The intentional leak may assist in purging carbon dioxide from the system during expiration to minimize the volume of carbon dioxide that may be re-breathed.

To produce the desired flow of pressurized gas 103 within the delivery conduit 109, the blower 101 may include a blower housing forming a volute containing an impeller or fan. An electric motor, such as a brushless DC motor, may couple to and rotate the fan. As the fan rotates, it draws gas (e.g., ambient air 111) in via an air inlet 104 of the blower housing where it is then compressed by the fan and expelled through the outlet 102 as a flow of pressurized gas 103. By controlling the rotational speed of the fan, the pressure of the flow of pressurized gas 103 within the delivery conduit 109 may be controlled to provide the desired treatment pressure to the user.

The apparatus 100 (e.g., the blower 101) may further include an electronic (e.g., microprocessor-based) controller that may, among other tasks, modulate or otherwise control a speed of the motor (and, accordingly, a speed of the fan), thereby regulating the treatment pressure and flow rate of the flow of pressurized gas 103. The controller and other components of the apparatus 100 may be powered by either an onboard power supply (e.g., a battery) or a remote power supply (e.g., AC or DC source).

While described and illustrated as a fan-based blower, the term “blower,” as used herein, may include any device capable to delivering pressurized gas to the delivery conduit. For example, the blower could also be a tank or bottle of compressed gas that is metered by a valve to provide the appropriate pressure and flow.

During operation of the apparatus 100, acoustic noise (sound energy (i.e., pressure) travelling as waves through air or other gases) produced by the blower 101 and the resulting flow of pressurized gas produced thereby may propagate through and along the delivery conduit 109. This acoustic noise may be bothersome to some users and, for certain users, may even interfere with the ability to sleep. The terms “acoustic noise,” “noise,” and “sound,” may be used interchangeably herein.

To address this issue, a muffler 200 defined by a tubular member or housing in accordance with embodiments of the present disclosure may be provided. As shown in FIGS. 1 and 2, the muffler may be an inline muffler that is operatively positioned between the blower 101 and the user interface 108. For example, as shown in FIGS. 1 and 2, the muffler 200 may be positioned in the gas delivery path between the blower 101 and the delivery tube 106.

Broadly speaking, the muffler 200 may define an expansion chamber within the delivery conduit 109. The muffler/expansion chamber may provide baffles (e.g., inwardly extending baffles) adapted to attenuate noise associated with the flow of pressurized gas as the gas passes through the muffler (such noise that would otherwise be detected downstream at the user interface). To achieve such noise attenuation, the baffles may be adapted to effectively capture sound waves (also referred to herein as sound energy or acoustic energy) associated with the flow of pressurized gas as the gas moves through the expansion chamber. As used herein, “capture” of sound energy may include most any baffle geometry that results in one or more of: destructive interference of sound energy; diffusion of sound energy; attenuation of sound energy; suppression of sound energy; absorption of sound energy; and redirection of sound energy. Mufflers in accordance with embodiments of the present disclosure may provide this capture function by configuring the baffles to interact with the flow of pressurized gas (as the gas passes through the expansion chamber) as described herein. As used herein, sound energy associated with the flow of pressurized gas may include sound energy produced: by the flow of gas; by the blower; and by any other system components that introduce acoustic energy into the system upstream of the muffler.

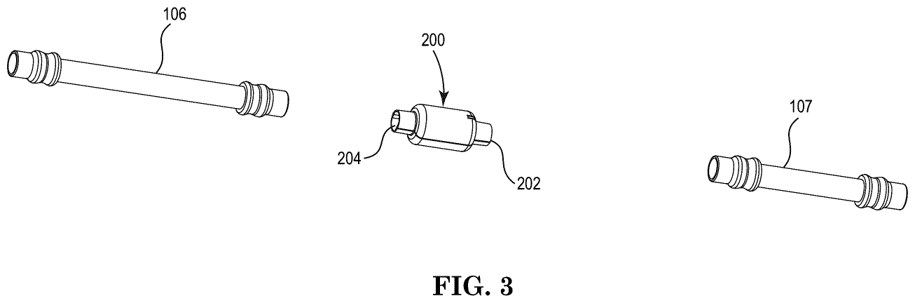

As stated above and shown in FIGS. 2-3, the muffler 200 may be operatively located between the blower 101 and the delivery tube 106. For example, the muffler housing may include a first end defining an inlet port 202 adapted to operatively couple to the blower outlet 102 (e.g., via an intermediate tubular hose 107), and a second end defining an outlet port 204 adapted to operatively couple to the proximal end of the delivery tube 106 that fluidly communicates with the user interface. The blower 101, hose 107, muffler 200, tube 106, and user interface 108 may be adapted to connect to one another as shown (see, e.g., FIG. 2) in a generally leak-free manner (other than any intentional leak provided on any one or more of these components).

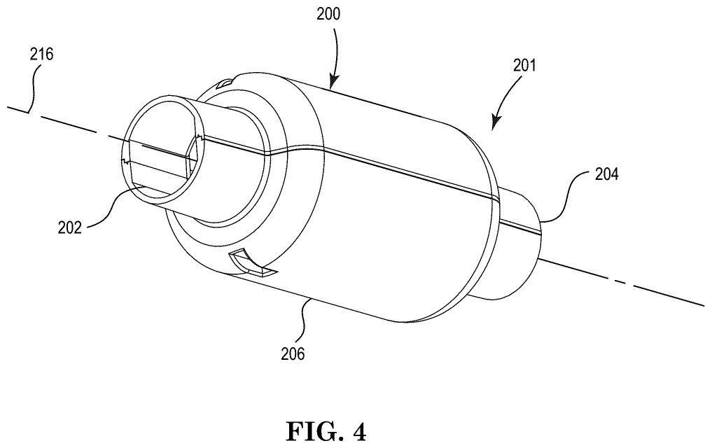

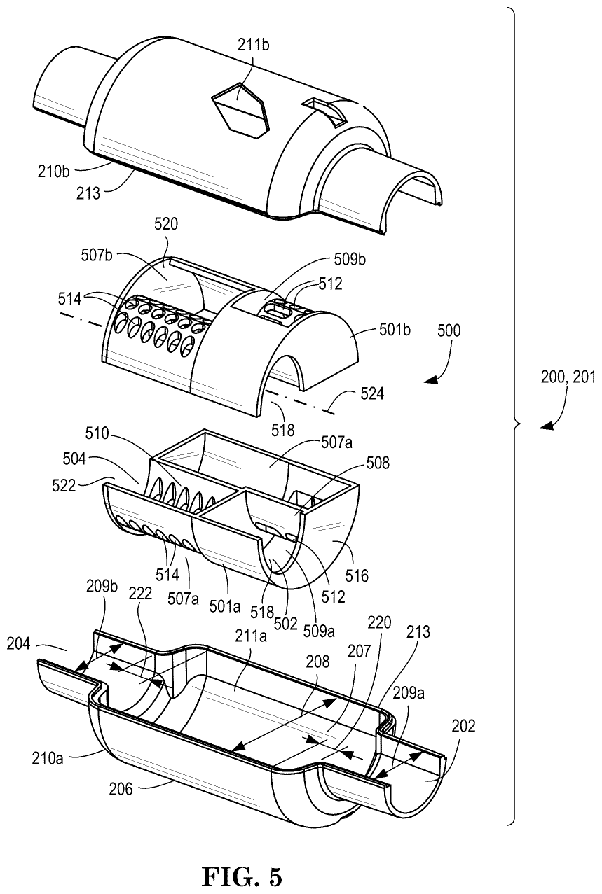

FIG. 4 is an enlarged perspective view of the exemplary muffler 200, while FIG. 5 illustrates an exploded view. As shown in these views, the inlet and outlet ports 202, 204 may define a common muffler axis 216 generally coaxial with a flow axis of the delivery conduit 109. Note that the muffler axis is “common’ in that it can serve as a common reference coordinate for the muffler 200 and any of its subassemblies. Moreover, the muffler 200/muffler housing may include a body 206 extending between the first and second ends (e.g., between the inlet and outlet ports 202, 204), wherein the body defines an expansion chamber 207 also located between the first and second ends of the housing. The expansion chamber 207 may include an effective cross-sectional area (e.g., as defined by an effective inner diameter 208) larger than an effective cross-sectional area of both the inlet port 202 (as defined by inner diameter 209a) and the outlet port 204 (as defined by inner diameter 209b).

While referred to herein as “diameter” and “effective diameter,” the inlet port 202, outlet port 204, and expansion chamber 207 may have most any cross-sectional internal (and external) shape without departing from the scope of this disclosure. That is to say, these terms may be used to refer to most any dimension associated with a cross-sectional geometry whether such geometry is circular or not. For example, the term “diameter” may be used to refer to a polygonal cross-sectional dimension, or an elliptical, oblong, or obround cross-sectional dimension without departing from the scope of this disclosure.

With reference still to FIG. 5, the exemplary muffler 200, the muffler housing 201 may include or be defined by two halves 210a, 210b (referred to individually and collectively as half or halves 210), each half (and thus each baffle) may be formed via a plastic (or other impermeable material), injection molding process. In the illustrated embodiment, each half 210a-b is identical, such that the muffler 200 may yield manufacturing economies as compared to alternative constructions by requiring only a single mold (or common mold parts or sections) to form the halves 210. The two halves 210 may be adapted to join or secure with one another along a generally planar mating surface 213 (see FIG. 5). Note that the actual mating surface may comprise stepped or curved surfaces, in which case the mating “surface” or mating “plane” may refer to a virtual or construction plane or surface about which the halves are symmetric when assembled. In other embodiments the two halves 210 may be symmetric in most pertinent respects yet may include different mating surface 213 configurations.

Such variation in the mating surfaces 213 of the two halves 210 may, for example, assist with joining (e.g., ultrasonic welding of) the two halves to one another to form the muffler 200. However, even with these mating surface variations, most or all other aspects of the halves—e.g., inner surfaces 211 of the halves 210, expansion chamber 207, etc., may be symmetric.

Accordingly, as used herein with respect to describing the halves 210, “symmetric” and like terms refer to structural symmetry of those features and components of the two halves that provide the primary acoustic noise capture mechanism (together with an insert as described below) and not necessarily those aspects related to joining the two halves to one another. Similarly, different features may be formed on or within the halves 210 in a separate process (e.g., stamping, drilling, bonding) that occurs after the initial forming process (e.g., injection molding). These features, such as external mount features, vent holes, or the like, do not affect the symmetry of the halves 210 as described above, in that a single injection mold can still be used to form both halves 210.

Also note that the term “halves” is mean to include separate interfacing parts that are roughly similar not intended to mean exactly half, e.g., that the pieces designated as halves each constitute 50% of the total assembly by weight or volume. For example, two parts formed from the same mold may be processed differently (e.g., molded of different density materials) and or post-processed differently (e.g., one has a hole drilled in it while the other does not), such that their weights and/or volumes are different. Also, a third component could be introduced into the assembly that is separate from the halves, e.g., an alignment pin, a fastener, a gasket, etc., such that the halves (e.g., halves 210) each constitute less than 50% of the entire assembly (e.g., the housing 201).

As shown in FIG. 5, the muffler 200 includes a muffler insert 500 positioned within the tubular body 206 of the housing 201 in an abutting relationship with inner surfaces 211a, 211b (referred to individually and collectively as surface or surfaces 211) of the body 206. The upper half 210b of the housing 201 is drawn with a cutaway to show the surface 211b. The insert 500 may directly contact the body 206 and/or intermediate structures (e.g., compliant pads or spacers) may be disposed between the insert 500 and body 206. The insert 500 and housing 201 may include interfacing features to locate and/or retain the muffler insert 500 within the housing 201, such as interfacing pegs/depressions, ridges/grooves, etc.

The muffler insert 500 defines/includes a first chamber 502 proximate the inlet port 202 and a second chamber 504 proximate the outlet port 204. An intermediate chamber 506 separate from, and located between, the first and second chambers 502, 504. A first wall 508 separates the first chamber 502 from the intermediate chamber 506 and a second wall 510 separates the second chamber 504 from the intermediate chamber 506. The first and second walls 508, 510 each define/include a plurality of apertures 512, 514 adapted to direct the flow of pressurized gas between adjacent chambers.

In this embodiment, the intermediate chamber 506 has a volume larger than a volume of either of the first and second chambers 502, 504. Note that the intermediate chamber 506 is open in regions 507a, 507b (referred to herein as open region or open regions 507) and the first chamber 502 is open in regions 509a, 509b (referred to herein as open region or open regions 509). These open regions 507, 509 can be considered a break or void in the outer wall of the intermediate chamber 506 and first chamber 502 respectively.

In this example, the open regions 507, 509 expose respective convex sides of the second and first walls 510, 508, the convex sides being outward facing with respect to the respective second and first chambers 504, 502. The open regions 507, 509 act as a mold passthrough so that an injection mold can form the second and first walls 510, 508. The open regions 507, 509 can thereby simplify manufacturing, e.g., allowing each of the insert halves 501a, 501b to be formed as single, injection molded piece. For other manufacturing modes, e.g., 3D printing, the open regions may not be required, as the insert can be formed, for example, on a build plane normal to the chamber axis 524. When assembled into the muffler housing 201, the inner surfaces 211 of the housing 201 seal the open regions 507, 509, such that the inner surfaces 211 form part of the intermediate chamber 506 and first chamber 502, respectively.

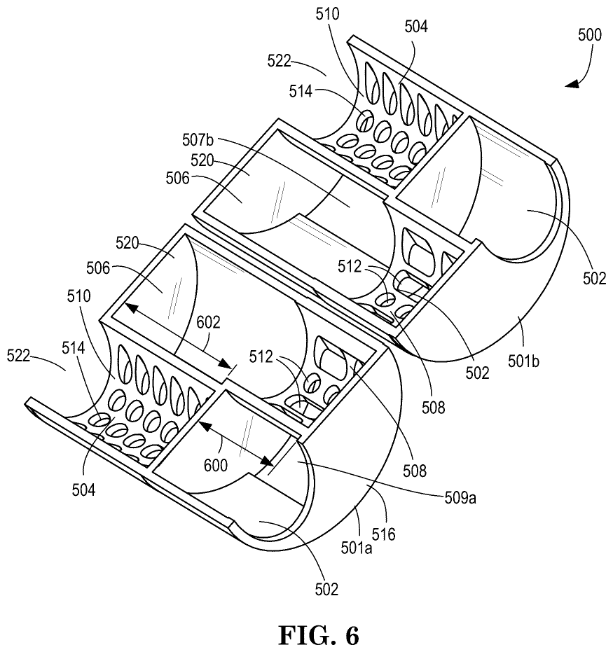

The muffler insert 500 includes a first external orifice (also referred to herein as an air inlet 518) and a second external orifice 522 (also referred to herein as an air outlet 522) that are respectively fluidly coupled to the input and output ports 202, 204 of the muffler housing 201. The air inlets and outlets 518, 522 are offset from the input and output ports 202, 204 so as to be able to form a convoluted gas passage as described elsewhere herein. This is indicated in FIG. 6 as chamber axis 524, which is parallel to and offset from muffler axis 216 (see FIG. 4).

When the muffler insert 500 is sandwiched between the housing halves 210, features of the housing (e.g., filleted corners of the input and output ends of the body 206) will enforce a clearance 220 between the muffler insert 500 and the input port 202. A corresponding clearance 222) is maintained between the muffler insert 500 and the output port 202. These clearances 220, 222 prevent flow restrictions into and out of the muffler insert 500, and may be achieved by other means, e.g., protrusions into an interior surface of the body 206.

The muffler insert 500 includes two halves 501a, 501b (referred to individually and collectively as insert half or insert halves 501) that are mirror images of one another, best seen in FIG. 6 where the insert halves 501 are shown side by side with interface surfaces facing upward. Because the halves 501 are mirror image and not symmetric, different molds may be used to form the two halves 501a-b where injection molding is used to form the insert 500. As best seen in FIG. 6, the open regions 507a-b allow each of the halves 501 be formed as a single molded piece. Note that, because different molds are used, the configuration of the halves 501 as mirror images of each other will not necessarily provide manufacturing efficiencies compared to symmetric design of the housing 201. The mirror image configuration may still have some advantages, e.g., ease of design and assembly. Nonetheless, non-mirror image halves 501 are also contemplated.

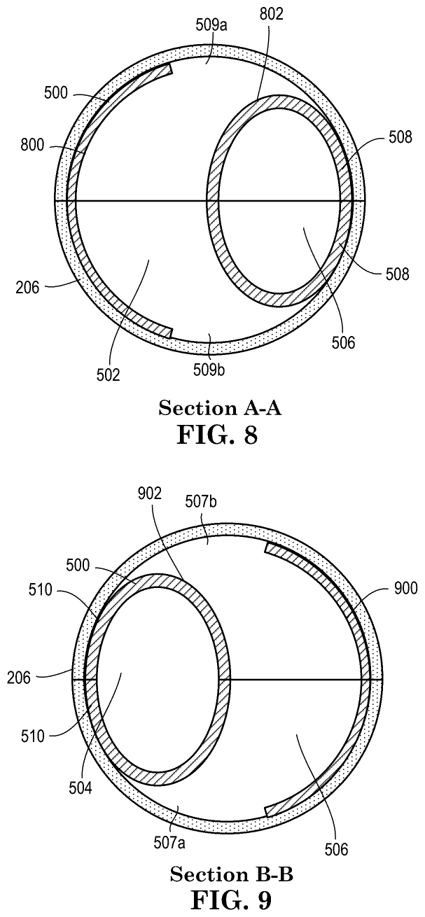

As seen in FIG. 6, the first chamber 502 has a dimension 600 along the muffler axis direction (see axis 216 in FIG. 4) that is smaller than a corresponding axis-aligned dimension 602 of the second chamber 504. This does not necessarily mean that a volume encompassed by the first chamber 502 is smaller than a volume encompassed by the second chamber 504. The different cross-sectional areas of the chambers 502, 504 (see FIGS. 8 and 9) permit the chambers 502, 504 to have similar or same internal volumes. In particular, the first chamber may have a larger cross-sectional area than that of the second chamber. Note that a size or number the apertures 512 may be larger than that of apertures 514 to account for the different chamber dimensions 600, 602, e.g., such that the resistance to gas flow is the same or similar across the first and second 508, 510 walls. This can serve to reduce a difference in gas flow resistance between the first and second 508, 510 walls.

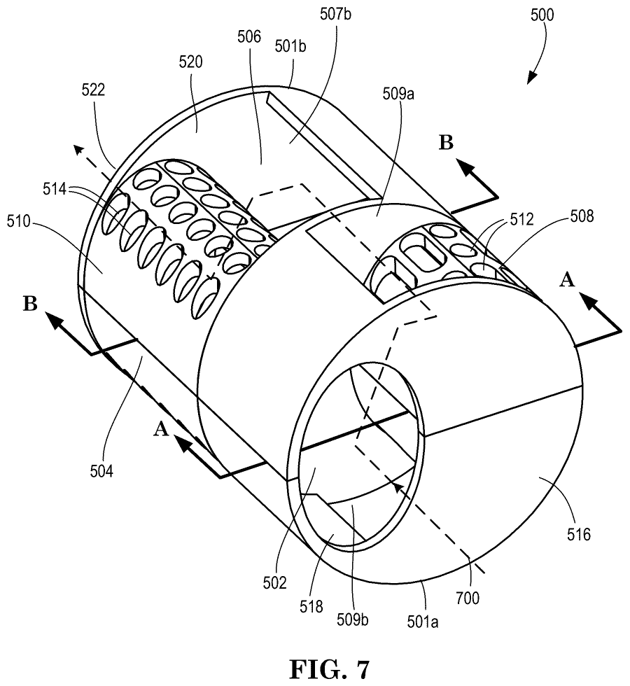

In FIG. 7, a perspective view shows the two halves 501 of the muffler insert 500 after being assembled. In this embodiment, the assembled muffler insert 500 has a generally cylindrical shape, within an inlet facing wall 516 and an outlet facing wall 520. The inlet wall 516 includes/defines an air inlet 518 which receives air from the inlet port 202 of the muffler housing 201. The outlet wall 520 includes/defines an air outlet 522 which expels air to the outlet port 204 of the muffler housing 201. The air outlet 522 can best be seen in FIGS. 5 and 6. Note that the descriptions of these orifices as being “inlets” and “outlets” is for purposes of convenience to describe one or illustrated embodiments, and as explained below, this is not meant to limit the function of these orifices when used in different muffler embodiments. Therefore, in other embodiments, the air inlet 518 may be considered a first external orifice of the first chamber 502 and the air outlet 522 may be considered a second external orifice of the second chamber 504.

The air inlet and outlet 518, 522 are aligned with one another along the muffler axis direction, however airflow from the air inlet and outlet 518, 522 takes a circuitous route through the chambers 502, 506, 504, respectively, as indicated by dashed line 700. Note that an upward facing set of the apertures 512, 514 are seen in this view, and there are a mirrored set of downward apertures 512, 514. This provides a second airflow pathway which would generally resemble a mirror image of line 700.

Generally, these circuitous routes through the muffler insert 500 help to reduce noise detected downstream of the muffler (e.g., at the user interface 108 shown in FIG. 2). This noise reduction can be achieved by disrupting gas flow, dampening of certain frequency ranges via acoustic resonance, causing flow to be turbulent, etc. For example, each of the chambers 502, 504, 506 will have one or more natural resonances for which sound at some frequencies will be amplified and sound at other frequencies will be reduced/attenuated. Therefore, the geometry of the chambers 502, 504, 506 (which includes the size and number of apertures 512, 514) can be selected to reduce particular frequency bands of interest. These resonant frequency bands can be determined using acoustic theory known in the art, and may be accurately modeled for complex geometries using computer simulation tools such as finite element analysis and computational fluid dynamics.

While the muffler insert 500 has orifices that may be specifically described air inlet and outlets 518, 522, the function of the these orifices may be reversed while still realizing the sound-attenuating benefits described above. For example, the muffler insert 500 may be deployed in reverse orientation relative to the inlet and outlet ports 202, 204 of the muffler housing 201 than what is shown in FIG. 5. In another example, the muffler insert 500 (in any orientation relative to the muffler housing 201) may be used to reduce noise in a bidirectional gas flow, such that the orifices may serve as both inlets and outlets depending on the gas flow direction.

As noted above, the chambers 502, 504, 506 have different cross-sectional geometries in a plane normal to the muffler axis. In FIGS. 8 and 9, cross-sectional views of the muffler insert 500 are shown which correspond to section lines A-A and B-B in FIG. 7. Note that in these cross-sectional views, the body 206 of the muffler housing 201 is shown surrounding the muffler insert sections such that the chambers 502, 504, 506 are fully enclosed in an assembled and operational configuration of the muffler 200.

The cross-sectional view of FIG. 8 shows the shapes of the first chamber 502 and part of the intermediate chamber 506. Due to the circular cross-sectional shapes of the muffler 200 in this embodiment and the oval shape of the walls 508, the first chamber 502 is generally crescent-shaped, with a circular convex edge 800 and an ovular concave edge 802. This part of the intermediate chamber 506 has an ovular-cross section. The second part of the intermediate chamber 506 is seen in FIG. 9 having a generally crescent cross-section shape, with a circular convex edge 900 and an ovular concave edge 902. The second chamber 504 has an ovular-cross section, which also defines the output shape of the air outlet 522.

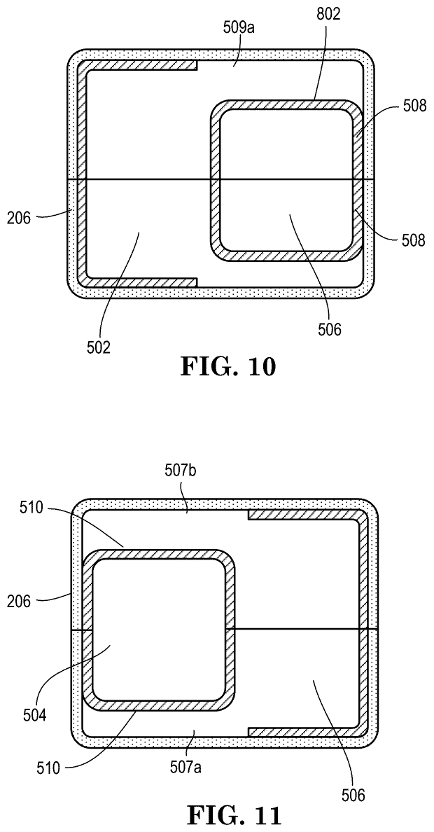

It will be understood that the cross-sectional shapes shown in FIGS. 8 and 9 are only one example of shapes of the muffler structure. For example, the circular cross section of the muffler housing body 206 and insert 500 may be made ovular instead of circular as shown without significant deviation of the shape of the part of the intermediate chamber 506 shown in FIG. 8 and the shape of the second chamber 504 shown in FIG. 9. In FIGS. 10 and 11, an example of a rounded rectangular cross section is shown that may be used instead of the curved shapes shown in other embodiments. While this embodiment may not share the manufacturability (e.g., need for injection molding draft angles for vertical surfaces) and acoustic characteristics (e.g., standing waves) of other embodiments, this configuration may still perform adequately under expected operating conditions, e.g., low gas velocities. Note that the muffler housing 201 in such an environment may still transition to a circular profile at the inlet and outlet ports 202, 204 to enable interfacing with tubular shaped hoses 107, 106.

Note that the use of a circular body 206 for the muffler housing 201 allows the insert 500 to be placed in any rotational orientation relative to the housing 201. For example, in FIGS. 8 and 9 the seams between the two parts of the body 206 and muffler insert 500 are shown aligned, however the body 206 or insert 500 may be rotated by any arbitrary angle without affecting the function of the muffler assembly 200.

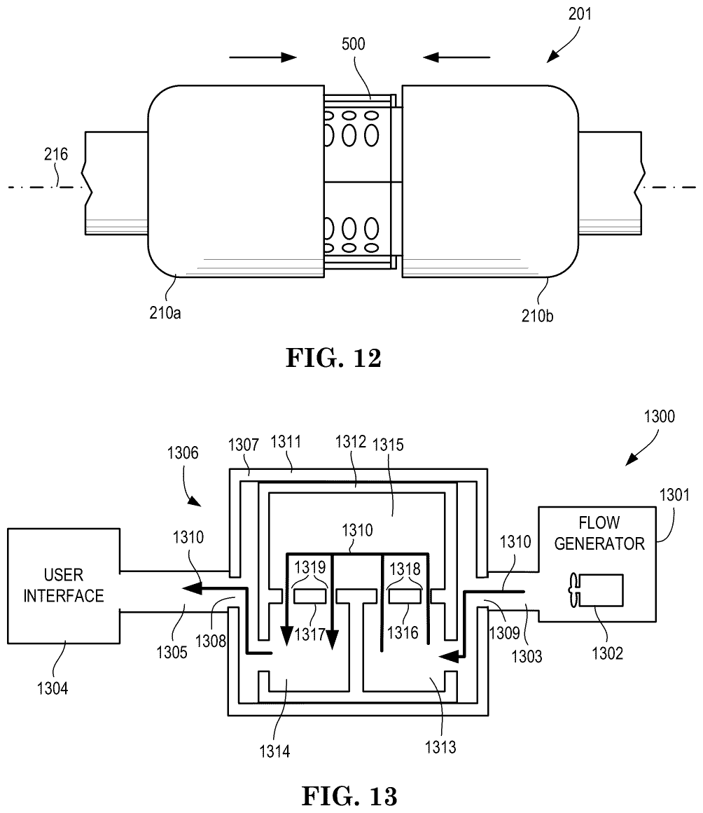

In the embodiments above, the muffler housing 201 is split into two parts, e.g., halves 210 seen in FIG. 5. Because the inside of the muffler insert 500 provides the air baffling functions, this allows alternate configurations of the muffler housing 201. In FIG. 12, a side view shows an alternate configuration of the muffler housing 201, in which two halves 210 of the housing 201 are split about a plane normal to the muffler axis 216. The halves 210 may still be symmetric about this plane, and may otherwise be configured and function as described for other embodiments of the housing 201. The halves 210 can be joined as indicated by the arrows to enclose the muffler insert 500. Note that the muffler housing 201 has three planes of symmetry. Two planes of symmetry as shown dividing housing halves 210 in FIGS. 5 and 12. The third plane of symmetry is the same as the plane of symmetry in FIG. 5, but rotated 90 degrees about the muffler axis. Therefore, a single mold (or common mold parts) could be used to create quarters or eights of the housing. This would result in a smaller mold but, among other things, could add time and complexity to the assembly.

In any of the embodiments described above, the muffler insert 500 may be permanently sealed inside of the muffler housing 201, e.g., by permanently affixing the halves 210 of the housing 201 using adhesives, ultrasonic welding, bonding, snap-fit engagement, fastening, overmolding, etc. In some cases, it may be desirable to allow the muffler housing 201 to be repeatedly opened and closed, e.g., via a user of the device. This could, for example, allow insertion or replacement of different muffler inserts 500 that are tuned for different acoustic performance by way of changing geometry of the chambers, flow apertures, changing of insert materials, etc. This may also allow the inside surfaces to be cleaned and disinfected by the user. Means for attaching and detaching the muffler housing halves 210 may include, but are not limited to, molded in snaps, fasteners (e.g., screws), clamping (e.g., a flexible band over the outside), magnets, etc. The insert 500 may also be separable, e.g., for cleaning, although may not require separate attachment and detachment means as the assembled housing halves 210 will also hold the halves of the insert 500, e.g., by sandwiching the insert between the housing halves 210.

In FIG. 13, a block diagram illustrates a positive airway pressure system and apparatus 1300. The apparatus includes a flow generator 1301 comprising a housing containing a blower 1302. The blower 1302 is operable to produce a flow of pressurized gas 1310 at a blower outlet 1303. The apparatus 1300 includes a user interface 1304 and one or more elongate delivery tubes 1305 positioned between the flow generator 1301 and the user interface 1304. The delivery tube(s) 1305 are operable to communicate the flow of pressurized gas 1310 from the blower 1302 to the user interface 1304.

An inline tubular muffler 1306 is positioned between the blower 1302 and the delivery tube 1305. The muffler 1306 is operable to attenuate noise associated with the flow of pressurized gas 1310 as the gas passes through the muffler 1306. The muffler 1306 includes a housing 1307 that defines an inlet port 1308 operatively coupled to the blower outlet 1303 and an outlet port 1309 operatively coupled to the delivery tube. The housing 1307 includes a tubular body 1311 extending between the inlet port 1308 and the outlet port 1309.

A muffler insert 1312 is positioned within the tubular body 1311 in an abutting relationship with inner surfaces of the body 1311. The muffler insert 1312 defines a first chamber 1313 proximate the inlet port 1309 and a second chamber 1314 proximate the outlet port 1308. An intermediate chamber 1315 is separate from, and located between, the first and second chambers 1314. For purposes of this embodiment, the term “between” is meant in the sense that the gas flow 1310 moves from the first chamber 1313 to the second chamber 1314 via the intermediate chamber 1315 and is not construed to mean that the intermediate chamber 1315 physically separates the first and second chambers 1313, 1314.

The insert includes a first wall 1316 separating the first chamber 1313 from the intermediate chamber 1315 and a second wall 1317 separating the second chamber 1314 from the intermediate chamber 1315. The first and second walls 1316, 1317 each define a plurality of apertures 1318, 1319, respectively, operable to direct the flow of pressurized gas between adjacent chambers 1313-1315.

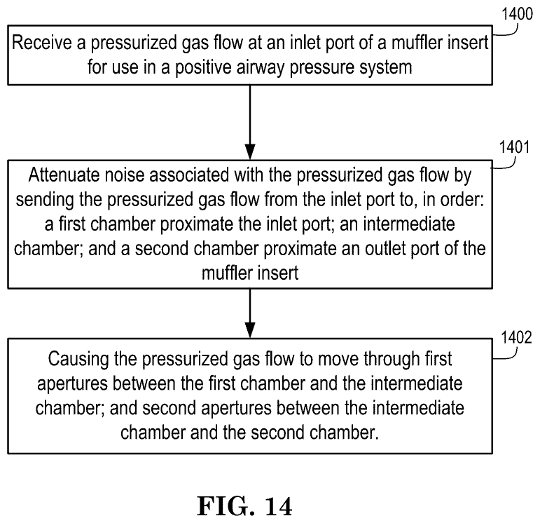

In FIG. 14, a flowchart shows a method according to an example embodiment. The method involves receiving 1400 a pressurized gas flow at an inlet port of a muffler insert for use in a positive airway pressure system. Noise associated with the pressurized gas flow is attenuated 1401 by the pressurized gas flow being sent from the inlet port to, in order, a first chamber proximate the inlet port, an intermediate chamber, and a second chamber proximate an outlet port of the muffler insert. The pressurized gas flow moves 1402 through: first apertures between the first chamber and the intermediate chamber; and second apertures between the intermediate chamber and the second chamber.

All headings provided are for the convenience of the reader and should not be used to limit the meaning of any text that follows the heading, unless so specified. Moreover, unless otherwise indicated, all numbers expressing quantities, and all terms expressing direction/orientation (e.g., vertical, horizontal, parallel, perpendicular, etc.) in the specification and claims are to be understood as being modified by the term “about.” The term “and/or” (if used) means one or all of the listed elements or a combination of any two or more of the listed elements. The term “i.e.” is used as an abbreviation for the Latin phrase id est and means “that is.” The term “e.g.” is used as an abbreviation for the Latin phrase exempli gratia and means “for example.”

It is noted that the terms “have,” “include,” “comprise,” and variations thereof, do not have a limiting meaning, and are used in their open-ended sense to generally mean “including, but not limited to,” where the terms appear in the accompanying description and claims. Further, “a,” “an,” “the,” “at least one,” and “one or more” are used interchangeably herein. Moreover, relative terms such as “left,” “right,” “front,” “fore,” “forward,” “rear,” “aft,” “rearward,” “top,” “bottom,” “side,” “upper,” “lower,” “above,” “below,” “horizontal,” “vertical,” and the like may be used herein and, if so, are for the benefit of explanation and/or are from the perspective shown in the particular figure. These terms are used only to simplify the description, however, and not to limit the interpretation of any embodiment described.

Further, it is understood that the description of any particular element as being connected to coupled to another element can be directly connected or coupled, or indirectly coupled/connected via intervening elements.

The complete disclosure of the patents, patent documents, and publications cited herein are incorporated by reference in their entirety as if each were individually incorporated. In the event that any inconsistency exists between the disclosure of the present application and the disclosure(s) of any document incorporated herein by reference, the disclosure of the present application shall govern.

Illustrative embodiments are described and reference has been made to possible variations of the same. These and other variations, combinations, and modifications will be apparent to those skilled in the art, and it should be understood that the claims are not limited to the illustrative embodiments set forth herein.

What is claimed is:

1. A muffler for use in a positive airway pressure system, the muffler comprising:
a housing defined by two halves, wherein the housing defines:
an inlet port adapted to operatively couple to receive a pressurized gas flow;
an outlet port adapted to output the pressurized gas flow; and
a tubular body extending between the inlet port and the outlet port; and
a muffler insert positioned within the tubular body in an abutting relationship with an inner surface of the body, wherein the muffler insert defines:
a first chamber proximate the inlet port;
a second chamber proximate the outlet port; and
an intermediate chamber separate from, and located between, the first and second chambers;
wherein the insert comprises: a first wall separating the first chamber from the intermediate chamber; and a second wall separating the second chamber from the intermediate chamber, wherein the first and second walls respectively define first and second pluralities of apertures adapted to direct the pressurized gas flow between adjacent chambers, the muffler adapted to attenuate noise associated with the pressurized gas flow as the pressurized gas flow passes through the muffler.

2. The muffler of claim 1, wherein the intermediate chamber has a volume larger than that of either of the first and second chambers.

3. The muffler of claim 1, wherein the inlet port and the outlet port define a common muffler axis, and wherein the first and second chambers define a common chamber axis that is parallel to and offset from the muffler axis.

4. The muffler of claim 1, wherein the muffler insert comprises two insert halves.

5. The muffler of claim 4, wherein the two insert halves are mirror images of one another.

6. The muffler of claim 4, wherein the two insert halves are secured to one another via securing together the two halves of the muffler housing, the two insert halves being sandwiched between the two halves of the muffler housing.

7. The muffler of claim 4, wherein the muffler insert defines at least one open region that exposes an outward facing surface of at least one of the first and second walls, wherein the at least one open region is sealed the inner surface of the body in the abutting relationship of the muffler insert with the inner surface.

8. The muffler of claim 1, wherein the first chamber defines a crescent shaped cross section.

9. The muffler of claim 1, wherein the first and second walls define an oval-shaped cross section when viewed along an axis of the muffler.

10. The muffler of claim 1, wherein the muffler insert is reversible within the housing such that the first chamber proximate the outlet port and the second chamber is proximate the inlet port.

11. The muffler of claim 1, wherein the muffler housing comprises symmetric halves formed from a same injection mold.

12. The muffler of claim 1, wherein each of the two halves are produced through an injection molding process.

13. The muffler of claim 12, wherein the two halves are secured to one another via a process selected from the group comprising ultrasonic welding, bonding, snap-fit engagement, fastening, and overmolding.

14. The muffler of claim 1, wherein the muffler insert comprises an impermeable material.

15. The muffler of claim 1, wherein the inlet port and the outlet port define a muffler axis direction, and wherein the first chamber has a dimension along the muffler axis direction that is smaller than a corresponding dimension of the second chamber along the muffler axis direction.

16. The muffler of claim 15, wherein the first chamber has a larger cross-sectional area than that of the second chamber.

17. The muffler of claim 15, wherein a size or number of the first plurality of apertures is larger than that of the second plurality of apertures such that a difference in gas flow resistance between the first and second walls is reduced.

18. A positive airway pressure apparatus comprising the muffler according to claim 1, the apparatus comprising:
a flow generator comprising a blower, the blower adapted to produce the pressurized gas flow at a blower outlet;
a user interface; and
an elongate delivery tube positioned between the flow generator and the user interface, the delivery tube adapted to communicate the pressurized gas flow from the blower to the user interface wherein the muffler is positioned between the blower and the delivery tube.

19. A method comprising:
receiving a pressurized gas flow at an inlet port of a muffler insert for use in a positive airway pressure system;
attenuating noise associated with the pressurized gas flow by sending the pressurized gas flow from the inlet port to, in order:
a first chamber proximate the inlet port;
an intermediate chamber, and
a second chamber proximate an outlet port of the muffler insert, wherein the pressurized gas flow moves through:
first apertures between the first chamber and the intermediate chamber; and
second apertures between the intermediate chamber and the second chamber.

20. The method of claim 19, further comprising:
producing the pressurized gas flow via a blower coupled to the inlet port; and
communicating the pressurized gas flow from the muffler insert to a user interface of the positive airway pressure system.
